(19) United States
(12) Patent Application Publication
(10) Pub. No.: US 2013/0334083 A1
(43) Pub. Date: Dec. 19, 2013

(54) CASSETTE FOR STORAGE OF MEDICAL INSTRUMENTS

(71) Applicant: Straumann Holding AG, Basel (CH)

(72) Inventors: Guillaume BUGNARD, Basel (CH); Joachim NAFZ, Waldachtal (DE); Klaus KANTORCZYK, Waldachtal (DE)

(73) Assignee: Straumann Holding AG, Basel (CH)

(21) Appl. No.: 13/923,718

(22) Filed: Jun. 21, 2013

(30) Foreign Application Priority Data
Dec. 23, 2010 (EP) 10 016 011.8

(51) Int. Cl. A61B 19/02

(52) U.S. Cl. CPC A61B 19/0271
USPC 206/370; 206/563

(57) ABSTRACT

Cassette for housing one or more medical instrument (18) including a tray having at least one passage (20) for receiving said medical instrument, said tray having a rigid structure (12) and a resilient holding means (28) arranged to retain the medical instrument (18) within said passage, wherein the holding means is located remote from the upper surface, and the at least one passage is formed by the rigid structure (12) and the holding means such that the uppermost section (44) of the passage is formed by the rigid structure and at least a portion of the lower section (46) of the passage is formed by the holding means.

### FIELD OF INVENTION

The invention relates to a cassette for storage and sterilization of at least one medical instrument, in particular dental surgical equipment.

### BACKGROUND

Cassettes for storage of medical instruments represent well-known systems for storage, organization, presentation and sterilization of said instruments. Most of these cassettes comprise at least one tray in which the medical instruments are accommodated in holding means, and a cover to close the tray.

Such a cassette is disclosed for instance in EP 08 016 174.8. Said cassette includes a tray and a cover for storage, transport and sterilization of medical instruments. The tray comprises holding means containing cutouts, which are attached to the basic structure of the tray in a gap free manner. In this way, bacteria, fluids, dirt etc. cannot enter into any intervening spaces between these two components. Thus the medical instruments can be stored under clean and sterile conditions for a long time.

Further examples of cassettes for storage of medical instruments are described in U.S. Pat. No. 5,384,103, U.S. Pat. No. 5,979,643, WO 2005/053597, WO 2006/071180 and U.S. 2007/0119737.

In the cassettes of the prior art, the holding means are fixed on the tray by form fit, molding, welding or gluing in order to have a connection which prevents the intrusion of the above mentioned substances. The cutouts, which hold the medical instruments in position, are intended to retain the medical instruments by a force-fit. These fittings have corners and edges and are therefore not easy to clean and sterilize for a further use of the cassette.

In some cases, the cutouts for holding medical tools are made of resilient, soft materials. Although such materials provide a good hold on the tool, these materials can also cause severe disadvantages. These materials are sensitive and prone to environmental pollution. In addition they can easily get contaminated with blood or soft tissue when the tools are grabbed by the surgeon with contaminated surgical gloves.

Dust, soft tissue and liquids like blood can easily lead to a contamination of materials of this kind which in addition are difficult to clean or sterilize.

In some cassettes of the prior art the resilient tool-holders are secured to the base of the tray by form fitting in corresponding openings of the tray. They are not, or not easily, removable which makes an effective cleaning procedure difficult or impossible. Because of this, fluids, bacteria or dirt which enters into gaps between the base and the resilient material may not be removed. Thus the sterility of the medical instruments might be at risk.

Even in case of removable resilient portions, these need to be pulled out of their recesses one by one, which in many cases requires the use of a specialized tool or a copped supporting means, which is labour-intensive.

### SUMMARY OF THE INVENTION

Therefore, it is an aim of at least a preferred embodiment of the present invention to provide a cassette which allows an optimized storage of medical instruments combined with the properties of being less prone to contamination and easily cleanable and sterilizable.

The cassette for housing one or more medical instruments according to the present invention comprises a tray having at least one passage for receiving said medical instrument, said tray comprising a rigid structure and a resilient holding means, said holding means being arranged to retain the medical instrument within said passage, wherein said holding means is located remote from the upper surface of the tray and the at least one passage is formed by the rigid structure and the holding means, such that the uppermost section of the passage is formed by the rigid structure and at least a portion of the lower section of the passage is formed by the holding means.

The present invention stems from the realization that an exposed arrangement of resilient material on the tray surface can be responsible for early contamination during utilization as well as the need for an enhanced cleaning effort.

In the present invention therefore, the holding means does not constitute the uppermost section of the passage and hence is not exposed on the upper surface of the tray.

Instead the holding means is fixed, either directly or indirectly, to the rigid structure at a location remote from the upper surface of the tray. Due to this arrangement the possibility of contaminating the holding means with blood or soft tissue is greatly reduced. When the surgeon grabs an instrument in the cassette with contaminated surgical gloves s/he does not contact the holding means. For the avoidance of doubt, the relative terms “upper”, “lower”, “top”, “bottom” etc as used within this specification refer to the orientation of cassette elements during use. In other words, the “upper” or “top” surface of the tray is that exposed during use and from which the surgeon or other user retrieves and replaces the stored instruments, while the “bottom” or “lower” surface is the surface which opposes this.

The holding means can be attached directly to the rigid structure via, e.g., moulding, bonding, gluing or mechanical means. Alternatively the holding means may be attached to the rigid structure indirectly via an intermediate part. When the holding means is attached to the rigid structure it is fixed in place relative to the rigid structure so that the rigid structure and holding means can be moved, e.g. into and out of the cassette, as a unit. This enables the instruments to be securely held by the tray even when this is removed from the cassette. In a preferred embodiment the holding means is removably attached to the rigid structure. It is therefore possible to remove the holding means in order to clean the parts individually. In addition the holding means can be replaced if these become contaminated or damaged. Therefore, if a contamination does occur, it is no longer necessary to exchange everything; it is possible to exchange only the holding means, which is of course less expensive.

In a preferred embodiment the holding means is directly attached to the rigid structure by form-fit engagement. This means that the rigid structure and holding means have complementary shapes which enable the parts to be securely connected simply pushing the parts together. This form of connection is beneficial as it does not require any additional or moving parts which would be disadvantageous in terms of cleaning and contamination risk. In addition, this type of connection increases the ease with which the holding means can be removed from the rigid structure. The form fit engagement can be a press fit connection, in which the pieces are held together by friction, or a snap fit connection, in which the pieces have complementary positive and negative interlocking elements, or a combination of both these connection types.

Preferably the form fit engagement comprises a snap fit connection wherein the holding means has a positive interlocking element and the rigid structure a negative interlocking element or vice versa. As mentioned above such a snap fit connection can be used alone or in combination with a press fit connection.

Preferably the form fit engagement is formed between one or more portions of the rigid structure and holding means, wherein other portions of these components are shaped so as to create channels between the two components. This allows sterilizing fluid (which can be a liquid or steam) to flow around and between the rigid structure and holding means, thus increasing the thoroughness of the sterilization process. The provision of channels between the rigid structure and holding means is beneficial in other embodiments also and therefore, more generally, the holding means and/or rigid structure may comprise grooves and/or ridges which, in use, form sterilization fluid channels between the components. Preferably the fluid channels are formed when the holding means and rigid structure are placed in a form fit engagement.

The resilient holding means can be formed from any material capable of retaining a medical instrument within the passage(s) of the cassette. Preferably the resilient holding means is made of a resilient plastic material which resists sterilization procedures, most preferably the holding means is made of silicon or a silicon based material or a thermoplastic elastomer (TPE).

The tray comprises at least one passage, typically vertically arranged, each passage intended for housing a single medical instrument or part of a medical instrument. In the case of large instruments or those with complex shapes more than one passage may be provided to house different parts of this instrument. Said passage(s) may be generally cylindrical or cone shaped, or comprise sections of both. In other words the diameter of the passage can remain constant, decrease or increase at various stages along its length. It is also possible for the diameter of the passage to undergo a step change. Such a step change forms an abutment surface within the passage which can act as a vertical stop for the medical instrument. The cross section of the passage can be any shape suitable for retaining a particular medical instrument, e.g. circular, oval, polygonal etc. Preferably the passage cross-section is circular. In the same or separate embodiment the passage is preferably cylindrical.

Although it is possible for the passage to comprise a closed passage, it is preferred that the at least one passage is a through hole. Thus, the passage extends from the upper surface to the lower surface of the tray. This allows flow of steam or liquid through the passage during sterilization processes. When a closed passage is provided the tray should further comprise side vents linking the passage to the tray exterior in order to ensure adequate flow of sterilization fluid.

The holding means can be arranged such that it forms only a part of the lower section of the passage, i.e. the holding means may be positioned such that a portion of the rigid structure or other tray component extends below it and forms the lowermost section of the passage. However, it is preferable that the lower section of the passage is formed entirely by the holding means. This enables the holding means to be exposed on the underside of the tray, which in turn aids removal and replacement of the holding means. Therefore, in a preferred embodiment the holding means is exposed on the underside of the tray. In such embodiments no part of the rigid structure or other part of the tray covers the underside of the holding means. This makes the holding means easily accessible during cleaning and maintenance of the cassette.

The holding means may form the lower five sixths, lower four fifths, lower three quarters, lower two-thirds, or lower half, of the passage. There is no limit to the percentage of the passage that can be formed by the holding means as long as a section of passage formed by the rigid structure exists above it. In this way the holding means does not form a part of the upper surface of the tray. This ensures that a contamination of the holding means by the surgeon is almost impossible, since there is no direct contact between the surgeon and the holding means during use of the tray. The minimum extent of the passage formed by the holding means is limited by the requirement that the holding means must be able to fulfill its function of retaining an instrument within the passage.

In a further preferred embodiment the holding means comprises at least one cutout for retaining the medical instrument, said cut out constituting the portion of the passage formed by the holding means. Said cutout may have a cross section corresponding to the cross section of the medical instrument and a diameter equal to or less than the instrument diameter in order to provide a secure hold. However, a tight fit between the instrument and the holding means around the entire circumference of the instrument would prevent flow of sterilizing fluid. Therefore it is preferable that the cutout comprises circumferentially spaced projections for retaining the medical instrument. These projections can run the entire length of the cutout, and in such embodiments it is equally valid to consider the cut out to comprise circumferentially spaced depressions. Alternatively the projections may cover only a partial section or intermittent sections of the cutout. The projections enable the holding means to firmly hold the instrument while at the same time providing channels within the passage for the flow of sterilization fluid. The projections are preferably evenly angularly spaced about the longitudinal axis of the passage. The projections can be any desired shape, for example they may have a rectangular, curved or triangular cross section. The projections may also assist with the central positioning of the medical instrument.

Preferably the holding means is arranged to retain the medical instrument by press-fit-engagement, which allows a fast removal of the instrument by the surgeon. It is however also possible for a snap fit engagement to be utilized.

As discussed above, the extent and shape of the passage formed by the holding means can vary depending on the design requirements. Design freedom is however limited by the need to ensure that the holding means is capable of retaining a medical instrument within the passage. It is preferable that, in use, the holding means provides a retentive force to the medical instrument of between 1 and 10 N.

Preferably, as discussed above, the holding means is removeable and exchangeable from the underside (lower surface) of the tray, although in some embodiments the holding means may be removeable and exchangeable from the sides of the tray. When the holding means is removeable from the underside of the tray, and the passage is through hole, it is preferable that the holding means comprises a passage counterbore on its underside. This enables a tool to be inserted into the counterbore to ease removal of the holding means. In alternative embodiments however a tool may be inserted directly into the through hole passage, or the underside of the holding means may comprise blind bores remote from the passage(s) for co-operation with such tools.

Preferably the tray comprises a plurality of holding means. These can be identical in shape or be provided in a variety of shapes depending on designer choice and needs. In a preferred embodiment a plurality of holding means is provided, each holding means comprising a plurality of cutouts. Thus each holding means forms two or more passages. Preferably the plurality of cut outs is arranged in a linear formation, i.e. in a straight line. In a particularly preferred embodiment a plurality of holding means is provided, each comprising three cutouts. This reduces the number of individual holding means, and thus the time needed to dis- and re-assemble the tray components, while still enabling targeted replacement of worn or damaged holding means. It is preferred that holding means designed to hold different instruments have different shapes. This assists the user in identifying the different holding means and thus with placing the correct holding means in the correct position on the tray. Alternatively or in addition holding means having different colours or distinctive markings, such as letters or numerals, can be provided. This is particularly beneficial in situations in which the holding means will be removed from the tray for sterilization and then later re-inserted. The different appearance of the holding means allows fast and accurate reassembly of the tray.

The rigid structure preferably comprises a hard, inflexible plastic material, such as PEEK, PPSU, PSU, PE, PP or PC although this could also be formed of metal, such as stainless steel. The uppermost surface of passage, and preferably the entire upper surface of the tray, at least in the vicinity of the at least one passage, is formed by the rigid structure. This structure is easier to clean and less susceptible to contamination than the resilient holding means. In order to assist with the cleaning process, it is preferable that the upper surface of the rigid structure is planar. A planar surface is easier to clean than one comprising grooves and protrusions, where dust and fluid can gather and which are difficult to clean effectively.

In another embodiment however, the rigid structure surrounding the passage slopes downwards away from the passage, such that the passage opening in the rigid structure is raised slightly from the planar surface. In this way, any liquid which lands on the tray in the vicinity of the passage will not flow into the passage, as it would if the surface sloped towards the passage, but will drain away from the passage. This helps to prevent contamination of the passage and the holding means.

Thus, more generally, in a preferred embodiment the rigid structure surrounding the passage opening is planar or slopes downwards away from the passage opening.

The rigid structure may be modular, i.e. the tray can be constructed from several independent rigid structures which are then welded, bonded or otherwise joined to form a gapless upper surface. This enables individualized trays to be created which have, for example, a particular passage layout or which are constructed from a variety of materials. However, preferably the rigid structure is integrally formed, e.g. by injection moulding. This simplifies manufacture and prevents any risk of creating contamination traps. Therefore in a preferred embodiment the rigid structure forms an integral, planar, gap free upper surface.

Preferably the underside of the rigid structure, i.e. the side opposite the upper surface, comprises at least one support structure which, in use, co-operates with the holding means to form a form fit engagement between the two components. Thus the support structure is a part of the rigid structure. Many co-operating support structures are possible, for example, the rigid structure may comprise downwardly extending pins with spherical heads for insertion into corresponding recesses within the holding means. Alternatively the support structure may comprise a cavity extending into the rigid structure for housing the holding means. When a plurality of holding means are provided for retaining a variety of different instruments it is preferable that the support structure for each type of holding means is distinctive. For example, the support structures may have different shapes, colours or markings (e.g. numerical or lettering). The corresponding holding means can be equivalently shaped, coloured or marked to ensure that, upon assembly or reassembly, the correct holding means is engaged which each support structure. Preferably the support structure for each type of holding means is geometrically distinct from other support structures, i.e. they have distinct shapes that ensure only the corresponding holding means and support structures can be placed in a form fit engagement. For example, unique arrangements of pins and recesses can ensure that only the correctly shaped holding means is attached to each part of the rigid structure.

In a preferred form the at least one support structure comprises a wall defining a compartment for housing a holding means. The compartment thus protects the holding means from damage as well as attaching this to the rigid structure. In this embodiment the compartment can also partially extend into the rigid structure. The wall may form a continuous, closed boundary around the compartment or a plurality of walls may form a partial boundary. In order for the holding means to be removeable it is preferred that at least one side of the compartment is open, i.e. it is not delimited by a wall. Preferably the compartment is open at its lower end, such that the holding means is exposed on the underside of the tray for easy removal. In this embodiment the at least one wall is a side wall. Thus in one preferred embodiment the at least one support structure comprises a side wall which forms a continuous, closed boundary around the compartment.

In one particularly preferred embodiment the rigid structure comprises a planar lower surface, from which a plurality of side walls protrude, the walls defining a plurality of compartments for housing the holding means.

The compartment and the holding means are shaped such that a form fit can be achieved. In a preferred embodiment the wall comprises at least one negative interlocking element for co-operation with at least one positive interlocking element of the holding means, or vice versa, such that a snap fit engagement can be achieved. In addition the interior surfaces of the compartment and/or the outer surface of the holding means can comprise grooves or ridges so that sterilization fluid channels are formed between the engaged components. Preferably the holding means comprises grooves on its exterior surface. This is preferred as the resilient material of the holding means is easier to shape than that of the rigid structure and by forming grooves in the resilient material sterilisation channels can be formed by placing these grooved holding means in contact with planar sections of the compartment walls.

Preferably the section of the passage formed by the rigid structure is dimensioned such that there is no or minimal contact between the rigid structure and the medical instrument housed within the passage. This enables a good flow of sterilising fluid past the housed instrument. However, in order to shield the holding means from fluid and other contaminants that may fall on the upper surface of the tray, the section of passage formed in the rigid structure should be kept as narrow as possible. In embodiments in which the holding means comprises a cut out with circumferentially spaced projections, the radius of the section of passage formed by the rigid structure should be greater than the minimum radius of the corresponding cut out, i.e. the projections should protrude into the passage defined by the rigid structure. However, it is preferred that the radius of the section of passage formed by the rigid structure is no greater than the maximum radius of the cut out of the corresponding holding means, i.e. the holding means which forms a section of the same passage. Thus, the radii of the holding means and rigid structure passage sections will be, in this embodiment, very similar. In this way, only the projections, or part of the projections, of the holding means are exposed through the rigid structure passage section and the remainder of the holding means is fully protected.

In an alternative embodiment, the section of the passage formed by the rigid structure can comprise circumferentially shaped protrusions, in the same manner as the cut outs of the holding means. However this is less preferred as the rigid structure may scratch or otherwise damage the medical instrument during insertion and removal.

The rigid structure forms at least the uppermost portion of the passage, that is, the section of the passage intersecting with the upper surface of the tray. In this way the holding means is kept remote from the surface of the tray, as this surface is most likely to encounter contamination. Preferably the rigid structure forms at least the uppermost 0.5 mm, preferably at least the first 1 mm of the passage.

Preferably the cassette comprises at least one medical instrument held in the at least one passageway. The medical instrument is preferably a dental surgical instrument and may be for example a drill, tap, depth gauge, sleeve, implant adapter or ratchet. Preferably the tray comprises a plurality of passages for housing a plurality of medical instruments. As mentioned above, the instruments can be held in a single passageway or may be held by a plurality of passageways.

In a particularly preferred embodiment the cassette houses a plurality of medical instruments and comprises a tray having a plurality of passages each suitable for receiving a medical instrument, the tray comprising a rigid structure having an upper surface and an underside which comprises a plurality of support structures, preferably in the form of walls defining compartments. The tray further comprises a plurality of resilient holding means arranged to retain said medical instruments within said passages, wherein the upper surface of the tray is formed by said rigid structure and the holding means are directly attached to the rigid structure at a location remote from the tray surface by means of a form fit engagement with the support structures.

The cassette may further comprise a base and a lid. The base comprises a bottom surface and four side walls. The tray can be arranged to fit over the top of the base (effectively sealing the base) or inside the base at a determined distance from the bottom surface (effectively sealing the volume of base below the tray). Alternatively the tray may be sized to fit over or in the base in a non sealing manner. This enables a plurality of trays to be placed at the same level within the tray. Generally the tray is orientated parallel to the bottom surface. The tray is preferably fixed to the base in a detachable manner. The base may house a plurality of trays. In addition the cassette may further comprise an intermediate portion located between the base and the lid onto which can be arranged a further tray. The lid may be attached to the base (or intermediate portion) by hinges or clamps etc. When the lid is hinged this may further comprise a tray in accordance with the present invention. During storage this tray will face the base of the cassette. However, when the cassette is opened for use the tray will have the same orientation as a tray in the base, that is, with the upper surface facing the user.

The preferred features of the invention listed above, except where obviously mutually exclusive, can be used in combination with any number of the other preferred features.

### BRIEF DESCRIPTION OF THE DRAWINGS

Particularly preferred embodiments of the present invention are described below, by way of example only, and illustrated in the drawings in which purely schematically:

FIG. 1a shows a first embodiment of the inventive cassette where a rigid structure and a holding element are presented in a lateral cut;

FIG. 1b shows a second embodiment of the inventive cassette where a rigid structure and a holding element are presented in a lateral cut;

FIG. 2 shows in a perspective view part of the bottom side of a rigid structure in accordance with the present invention;

FIG. 3 shows a perspective view of a holding means according to the present invention having three cutouts;

FIG. 4 shows in a perspective view the rigid structure of FIG. 2, containing the holding means of FIG. 3;

FIG. 5 shows a cross section through line I-I of FIG. 4;

FIG. 6 shows a perspective view of a cassette according to the present invention;

FIG. 7 shows the tray of FIG. 6 in isolation from a top perspective view; and

FIG. 8 shows the tray of FIG. 7 from a bottom perspective view.

### DETAILED DESCRIPTION

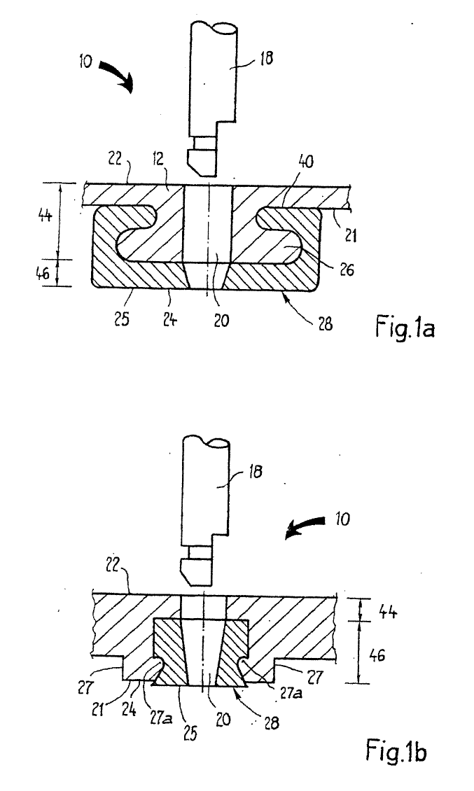

FIGS. 1a and 1b show detailed views of first and second embodiments of the present invention. The tray 10 comprises a rigid structure 12 and a holding means 28. The holding means 28 is intended to retain a medical instrument 18. Said medical instrument 18 may be any instrument which is used during an implant operation such as drills, taps, depth gauges, sleeves, implant adapters, ratchets. At least one passage 20 is formed in the tray 10, and in most cases a plurality of passages 20 are formed. Each passage 20 is designed for receiving a single medical instrument 18 or part of a medical instrument 18. The passages 20 are preferably distributed over the entire surface of the tray 10 in order to maximize the number of medical instruments 18 which may be stored in the cassette according to the present invention.

As shown in FIG. 1a and FIG. 1b, said passage 20 extends from an upper surface 22 to a lower surface (underside) 24 of the tray 10, thus forming a through hole. The upper surface 22 is formed entirely by rigid structure 12 and hence the uppermost section of passage 20 is formed in the rigid structure 12. In this way, the holding means 28 is less exposed to any contamination which may occur when the surgeon removes and later re-inserts the tool 18. Any blood, soft tissue or other substance falling onto the tray 10 at this time will only contact the rigid structure 12, which is easier to clean than the resilient holding means 28. The holding means 28 is removably fixed to the rigid structure 12 at a location remote from the upper surface 22, in this case to the underside of the rigid structure 12. This enables the holding means 28 to be easily removed and cleaned or exchanged in the case of contamination.

The passage 20 has an upper section 44 towards the top side 22 and a lower section 46 towards the bottom side 24, wherein the holding means 28 is arranged only in the lower section 46 of the passage 20.

In the embodiment shown in FIG. 1a, the holding means 28 forms only a small amount of said passage 20, whereas in the embodiment shown in FIG. 1b the holding means 28 forms the majority of the passage 20. There is no upper limit on the extent of the passage 20 which can be formed by the holding means 28 other than that this must not form part of the upper surface 22 of the tray 10. Therefore, there will always be an upper section 44 of the passage 20 located above the holding means 28 which is formed by the rigid structure 12. The lower limit of the extent of the passage 20 which can be formed by the holding means is dependent on the retentive force provided by the means 28. This must be capable of securely yet releasably retaining a medical instrument 18 within the passage 20. Although in the embodiments of FIG. 1 the lower section 46 is formed entirely by the holding means 28, it is possible for the rigid structure or some other structure to extend below the holding means, thus forming the lowermost part of the passage 20.

In the embodiments shown in FIG. 1 the holding means 28 is fixed to the rigid structure 12 by form-fit engagement on the underside 24 of the tray 10. In the embodiment of FIG. 1a the rigid structure 12 comprises a support structure 26 protruding from the underside 21 of the rigid structure. In the embodiment of FIG. 1b rigid structure 12 comprises a cavity defined by walls 27.

The holding means 28 is shaped to engage the corresponding support structure of the rigid structure 12. In both embodiments of FIG. 1 the holding means 28 and rigid structure 12 form a snap fit engagement. In the case of FIG. 1a the holding means 28 has an element with a positive shape which corresponds to the undercut 40 formed by support structure 26. Preferably said elements are round in order to ensure a good sterilization. In FIG. 1b the inside surfaces of walls 27 comprise positive elements 27a which correspond to negative indents in the holding means 28. In FIG. 1b a press fit connection is also made between the cavity surfaces and the holding means 28. Of course, a form fit comprising only a press fit connection could be used in the embodiment of FIG. 1b simply by removing the positive 27a and negative elements. In this case the complementary shapes of the holding means 28 and rigid structure 12 would be held in frictional engagement with one another. The frictional engagement can be strengthened by compression of the holding means 28 within the cavity.

In FIG. 1a the passage 20 is essentially cylindrical. Towards the lower surface 24 of the tray 10 the diameter of the passage 20 is tapered. However, the shape of the passage 20 depends on the medical instrument 18. Therefore it is also possible that said passage 20 has an essentially conical shape (as shown in FIG. 1b). Although in these embodiments the taper is shown as being formed in the holding means 28 only, it is of course also possible for a taper to be formed in the rigid structure or for the holding means to form a cylindrical section of the passage. In some cases a stop may be formed within the lower section 46 of the passage 20, onto which the instrument 18 can abut.

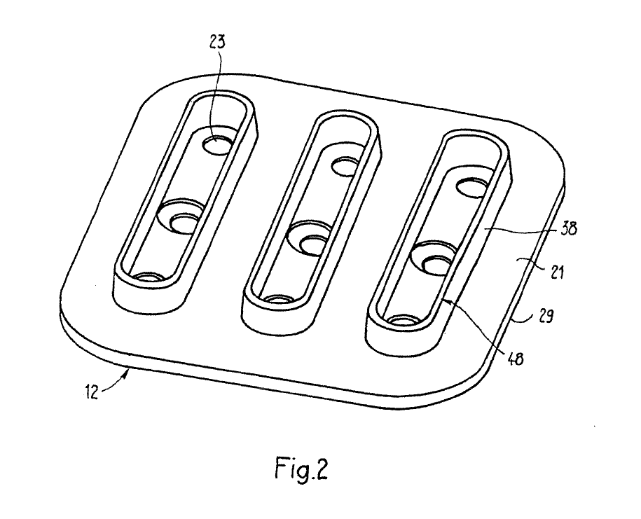

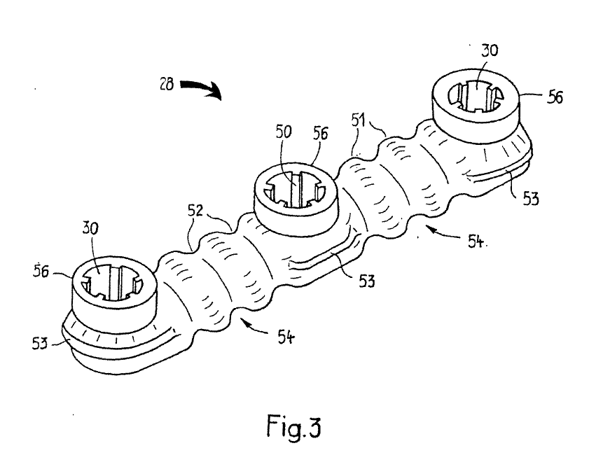

FIG. 2 shows a perspective bottom view of a rigid structure 12 according to a further embodiment of the present invention. The rigid structure 12 comprises several apertures 23 extending from the top surface 29 to the bottom surface 21 of said rigid structure 12. Top surface 29 forms the upper surface 22 of tray 10. The rigid structure 12 comprises three compartments 48, which are formed by side walls 38 extending downwardly from bottom surface 21. Each compartment 48 contains three apertures 23. Each compartment 48 is intended to receive the holding means 28 shown in FIG. 3.

FIG. 3 shows a preferred embodiment of the holding means 28, which is intended to be fixed to the rigid structure 12 shown in FIG. 2.

The body of said holding means 28 is linear and tubular. Preferably it is a single part made of one material which can be silicon, a silicon based material or a TPE. It comprises three cylindrical sections 56, each one containing a cutout 30. The cutouts 30 comprise circumferentially spaced projections in the form of rails 50. These rails 50 define an inner diameter corresponding to the cross section of a medical instrument 18 in order to provide a secure retention. The provision of rails 50 allows for the secure retention of the tool whilst forming channels between the rails 50 which allow flow of sterilizing fluid through the cut out 30. In addition rails 50 help to facilitate the positioning of the medical instrument 18. The cylindrical sections 56 are linked together by interconnections 54. These interconnections 54 consist of a number of ridges 51 separated by grooves 52.

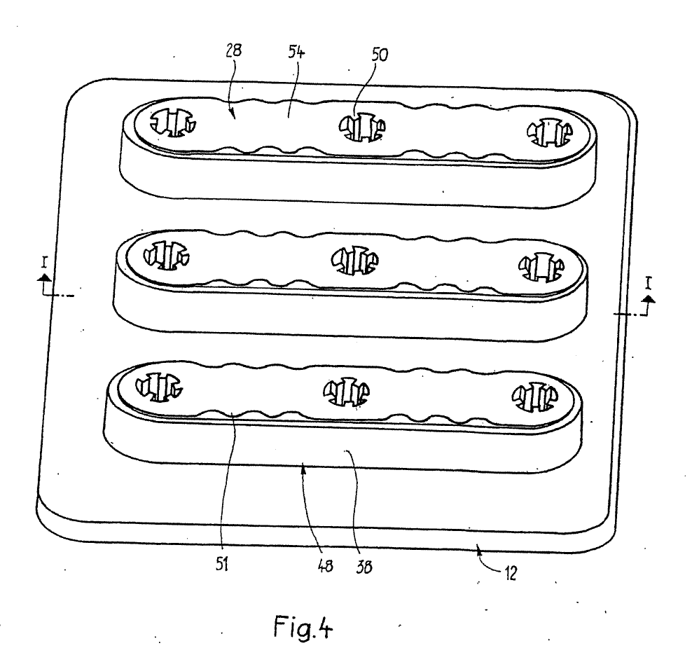

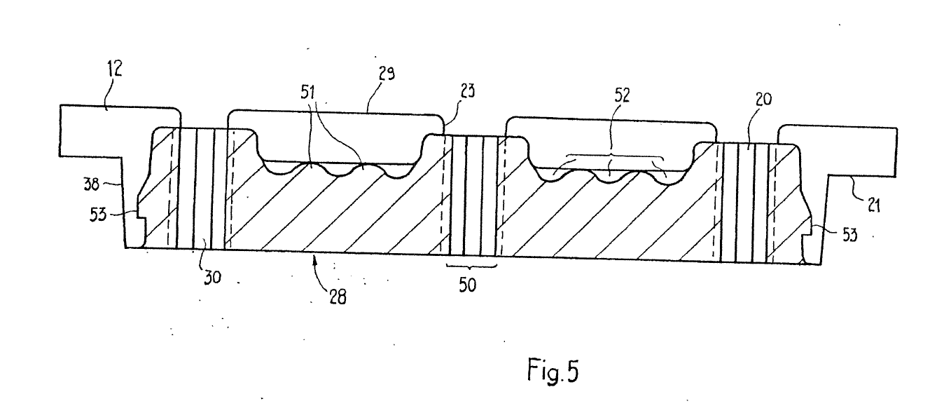

The holding means 28 is shaped to fit into the compartments 48 formed by the side walls 38 of rigid structure 12. This is shown in FIGS. 4 and 5. Cut outs 30 are arranged to align with apertures 23 in order to form passages 20. The outer surface of the ridges 51 and cylindrical sections 56 are fixed to the rigid structure 12 by press-fit engagement with the side walls 38. In addition, lips 53 having abutment surfaces are formed in the outer surface of holding means 28. These are housed within corresponding grooves in side walls 38 to form an enhanced snap fit engagement, as can best be seen in FIG. 5. Grooves 52 form channels between the holding means 28 and rigid structure 12 for the flow of sterilizing fluid.

As can be seen from FIG. 5, the radius of the apertures 23 in the rigid structure is slightly less than the maximum radius of cut outs 30 but greater than the minimum radius, defined by rails 50. In this way only the rails 50 are exposed through the apertures 23 and the remainder of the holding means 28 is protected.

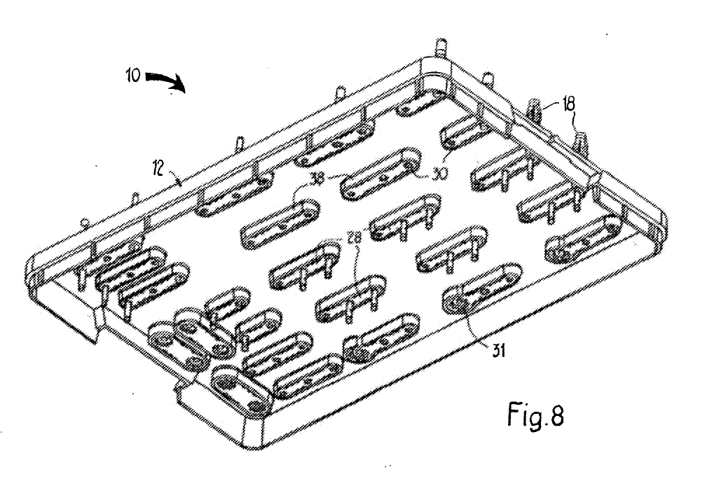

As is clear from these figures, cut outs 30 extend through the length of the holding means 28 to form through passages. In this embodiment the cross section of cut out 30 remains constant along the length of the holding means 28. However, in other embodiments the lower end of cut outs 30 may vary in cross section, in order to simplify the replacement of the holding means 28, e.g. by being shaped for co-operation with a particular tool (see FIG. 8). In some embodiments the cut out 30 may not extend through the length of the holding means 28. In such embodiments vent holes must be provided to allow for proper circulation of sterilizing fluid. These could connect the cut out 30 to the underside of the holding means 28 (therefore effectively forming a through passage) or to the sides.

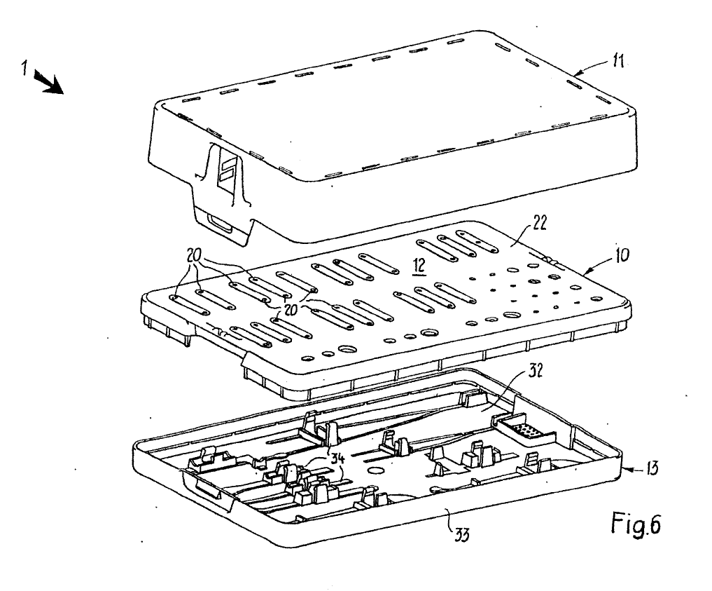

FIG. 6 shows a cassette 1 for the storage of medical instruments 18 comprising a tray 10, base 13 and cover 11. The tray 10 has basically a rectangular shape and may have rounded edges. The cassette can be closed with the cover 11 for the transport or the storage of the medical instruments 18. The cover 11 may be fixed to the base 13 by means of clamp-connection. The base 13 and cover 11 are preferably made of a metal or a rigid plastic material which can resist standard sterilization procedures and impacts from the outside. The base 13 or the cover 11 can be provided with anti-slip means in order to avoid slipping of the cassette if it is placed on slippery surfaces.

Base 13 comprises a bottom surface 32 and side walls 33. Bottom surface 32 comprises holding elements 34 for retention of medical tools. These do not form part of the present invention and so will not be discussed further.

Tray 10 is adapted to fit on top of base 13. Upper surface 22 of tray 10 is formed by rigid structure 12 and is produced by injection molding. This enables a unitary, planar upper surface 12 to be produced which is beneficial for cleaning purposes. Tray 10 contains a plurality of passages 20 for receipt of medical instruments 18. It is to be noted that the dark circles which appear around several of these passages 20 do not represent grooves or other irregularities on the upper surface 22, but instead indicate the position of side walls 38 on the underside of the rigid structure. It is possible however for the upper surface 22 of the tray to be painted or otherwise marked to assist the surgeon with tool identification.

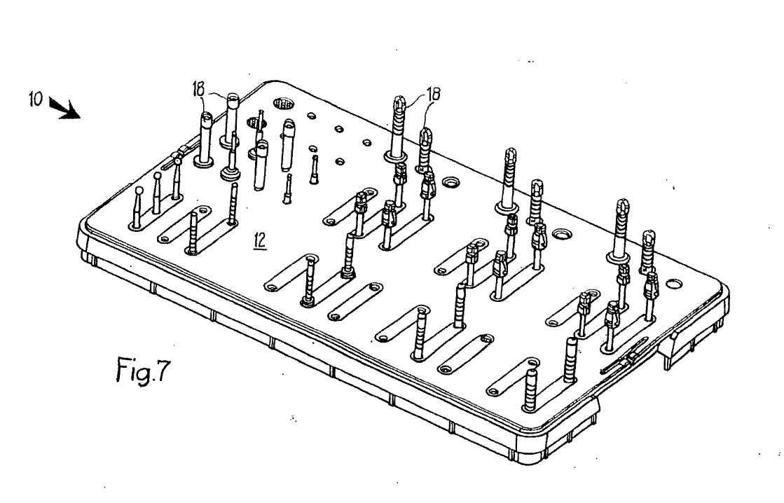

FIG. 7 shows the tray 10 of FIG. 6 in isolation. In this figure a number of instruments 18 are shown housed in passages 20.

FIG. 8 shows the underside of the tray 10 of FIG. 7. Here the side walls 38 can be clearly seen. Each side wall 38 defines a compartment within which a complementary shaped holding means 28 is housed in a form fit engagement. These holding means 28 have an identical or similar construction to the holding means of FIG. 3. Cut outs 30 within the holding means 28 form the lower section of passages 20. Rails 50 and grooves 52 ensure channels remain between the holding means 28 and instruments 18 and the holding means 28 and rigid structure 12 respectively, such that sterilising fluid can flow around these parts. This figure also demonstrates the ease with which the holding means 28 can be removed if necessary. The compartments 48 comprise side walls 38 only. Therefore each holding means is exposed on the underside of the tray and so can be pulled from the compartments 48 by inserting a tool into cut out 30. In addition, several of the cut outs 30 comprise a widened base part 31 (counterbore). This base part 31 of the cut out 30 is shaped to engage with a tool to assist removal of the holding means 28.

As can be seen in FIG. 8, the exact shape of each complementary holding means 28 and compartment can vary. This ensures that, following removal of the holding means for sterilisation purposes the right holding means is placed in each compartment. The holding means 28 could also be coloured or have an indentifying mark moulded onto the outer surface. A complementary coloured or marked compartment would ensure the correct pairing of holding means and support structures.

Therefore, according to the present invention, medical instruments can be reliably retained within a cassette by resilient holding means. The positioning of these holding means at a location remote from the upper surface of the tray reduces the contamination risk to these parts. In addition, having the holding means removeably fixed to the rigid structure and ideally exposed on the underside of the tray enables fast and easy removal and replacement of these pieces should contamination occur.

The above described embodiments are for illustrative purposes only and the skilled man will realize that many alternative arrangements are possible which fall within the scope of the claims. For example, the tray and base may be integrally formed. The holding means may take the form of single or merged grommets partially inserted into a passage formed by the rigid structure, such that the holding means lines the lower section of the passage. In addition many complementary shapes which enable form fit engagement of the holding means and rigid structure can be designed.

1. A cassette for housing one or more medical instrument comprising
a tray having at least one passage for receiving said medical instrument, said tray comprising
a rigid structure and
a resilient holding means arranged to retain the medical instrument within said passage,
wherein said holding means is located remote from the upper surface of the tray and the at least one passage is formed by the rigid structure and the holding means such that the uppermost section of the passage is formed by the rigid structure and at least a portion of the lower section of the passage is formed by the holding means.

2. A cassette as claimed in claim 1 wherein the holding means is removeably attached to the rigid structure.

3. Cassette according to claim 1, characterized in that the holding means is directly attached to the rigid structure by form-fit engagement.

4. Cassette according to claim 3, characterized in that the form fit engagement comprises a snap fit connection wherein holding means has a positive interlocking element and the rigid structure a negative interlocking element or vice versa.

5. Cassette as claimed in claim 1, wherein the holding means and/or rigid structure comprise grooves and/or ridges which, in use, form sterilization fluid channels between the components.

6. Cassette as claimed in claim 1, wherein the at least one passage is a through hole.

7. Cassette as claimed in claim 1, wherein the lower section of the passage is formed by the holding means.

8. Cassette as claimed in claim 7, wherein the holding means is exposed on the underside of the tray.

9. Cassette according to claim 7, characterized in that the holding means forms at least the lower two-thirds the passage.

10. Cassette according to claim 1, characterized in that the holding means comprises one or more cutout for retaining the medical instrument, said cut out comprising the portion of the passage formed by the holding means.

11. The cassette according to claim 10, characterized in that the one or more cut out is arranged to retain the medical instrument by press-fit engagement.

12. The cassette according to claim 10 wherein the one or more cut out comprises circumferentially spaced projections for retaining the medical instrument.

13. The cassette as claimed in claim 1, where in the holding means is arranged such that, in use, it provides a retentive force to the medical instrument of between 1-10N.

14. Cassette as claimed in claim 1, wherein the rigid structure surrounding the passage opening is planar or slopes downwards away from the passage opening.

15. Cassette as claimed in claim 1, wherein the upper surface of the rigid structure is planar.

16. Cassette as claimed in claim 1, wherein the lower surface of the rigid structure comprises at least one support structure which, in use, co-operates with the holding means to create a form fit engagement.

17. Cassette as claimed in claim 16, wherein the support structure comprises a wall defining compartment for housing the holding means.

18. Cassette as claimed in claim 17, wherein the wall is a closed, continuous side wall.

19. Cassette as claimed in claim 16, wherein the tray comprises a plurality of holding means and corresponding support structures for retaining a plurality of different instruments, wherein the support structure for each type of holding means is geometrically distinct from other support structures.

20. Cassette as claimed in claim 1, further comprising at least one medical instrument held in the at least one passageway.

21. Cassette as claimed in claim 20, wherein the cassette houses a plurality of medical instruments, the tray having a plurality of passages each suitable for receiving a medical instrument, the rigid structure having an upper surface and an underside which comprises a plurality of support structures in the form of walls which define a plurality of compartments, the tray further comprising a plurality of resilient holding means directly attached to the rigid structure by means of a form fit engagement with the support structures.

22. Holding means for a cassette according to claim 1, comprising a cutout and a positive element or a negative element intended to engage into the corresponding negative element or positive element of rigid structure.
